(19) United States
(12) Patent Application Publication
(10) Pub. No.: US 2002/0051983 A1
(43) Pub. Date: May 2, 2002

(54) Detection of amplified products in nucleic acid assays following nuclease treatment

(76) Inventor: Stuart Harbron, Berkhamsted (GB)

Correspondence Address: Hopgood, Calimafde, Judlowe & Mondolino, 60 East 42nd Street, New York, NY, 10165, US

(21) Appl. No.: 09/840,499

(22) Filed: Apr. 23, 2001

(30) Foreign Application Priority Data
Oct. 22, 1999 (GB) PCT/GB99/03510
Oct. 22, 1998 (GB) GB 9823005.5

(51) Int. Cl. C12Q 1/68; C07H 21/02; C07H 21/04; C12P 19/34

(52) U.S. Cl. 435/6; 435/91.1; 435/91.2; 536/23.1

(57) ABSTRACT

The present invention provides a method for detecting nucleic acid amplification product of a target-dependent nucleic acid amplification process involving one or more probes or primers, comprising the steps of: a) treating said product with a nuclease reagent whereby said product is substantially hydrolysed into its mononucleotide components; b) detecting said mononucleotide components. The amplification product is suitably selected for by use of a nuclease reagent that is specific for polyribonucleotide when said product is a polyribonucleotide, and is specific for polydeoxyribonucleotide when said product is a polydeoxyribonucleotide. Alternatively, the detection step may be specific for the mononucleotide components from the product by being selective between ribonucleotides and deoxyribonucleotides.

### FIELD OF THE INVENTION

This invention is concerned with nucleic acid amplification techniques, and is particularly directed at methods for detecting products of nucleic acid amplification procedures and is especially but not necessarily exclusively suitable for use in diagnostics including clinical diagnostics.

### BACKGROUND ART

Many methods for the detection of nucleic acids (DNA or RNA) have been developed. The more sensitive of these use amplification techniques to increase the number of copies of the target nucleic acid.

In U.S. Pat Nos. 4,683,195 and 4,683,202, DNA or RNA is amplified by the polymerase chain reaction (PCR). These patents are incorporated herein by reference in their entirety. This method involves the hybridisation of an oligonucleotide pnmer to the 5′ end of each complementary strand of the doublestranded target nucleic acid. The primers are extended from the 3′ end in a 5′-3′ direction by a DNA polymerase, which incorporates free nucleotides into a nucleic acid sequence complementary to each strand of the target nucleic acid. After dissociation of the extension products from the target nucleic acid strands, the extension products become target sequences for the next cycle. In order to obtain satisfactory amounts of the amplified DNA, repeated cycles must be carried out, between which cycles, the complementary DNA strands must be denatured under elevated temperatures.

A method of detecting a specific nucleic acid sequence present in low copy in a mixture of nucleic acids, called ligase chain reaction (LCR), has also been described. WO 89109835 describes this method and is incorporated herein by reference in its entirety. Target nucleic acid in a sample is annealed to probes containing contiguous sequences. Upon hybridisation, the probes are ligated to form detectable fused probes complementary to the original target nucleic acid. The fused probes are disassociated from the nucleic acid and serve as a template for further hybridisation's and fusions of the probes, thus amplifying geometrically the nucleic acid to be detected. The method does not use DNA polymerase.

Other known nucleic acid amplification procedures include transcription-based amplification systems (Kwoh et al., Proc. Natl. Acad. Sci. (U.S.A.) (1989) 86:1173; Gingeras et aL, WO 88/10315; Davey et a!., EP 329,822; Miller et aL, WO 89/06700), RACE (Frohman, In: PCR Protocols: A Guide to Methods and Applications, Academic Press, N.Y. (1990)) and one-sided PCR (Ohara, et aL, Proc. Natl. Acad. Sci. (U.S.A.) (1989) 86:5673-5677). Particularly suitable amplification procedures include Nucleic Acid Sequence-Based Amplification (NASBA, Transcription Mediated Amplification (TMA), Strand Displacement Amplification (SDA), and Cycling Probe Amplification.

Alternatively, a sequence in the probe or primer used may be amplified. Thus Cytocell Ltd (WO93/06240) has developed and isothermal amplification protocol, termed PEDIAT (Primer Extension Dependent Isothermal Amplification Technology). This approach utilises Klenow DNA polymerase and T7 RNA polymerase, and two oligonucleotide probes. Each probe has one region that can hybridise to the target and a shorter region that can hybridise to the other probe. The probes therefore only anneal in the presence of the target, forming a three-way junction structure. A double-stranded RNA polymerase promoter is either formed directly by the region of overlap between the two probes, or is created by Klenow extension. Multiple copies of RNA produced by the T7 RNA polymerase is detected using further probes, or if required, further cycled amplification. Another approach from Cytocell is disclosed in WO98/27225, which describes an approach called LOOT (Loping out of Target).

With all these techniques, it is necessary to detect the amplified nucleic acid when the amplification steps are complete: a number of different ways of achieving this have been developed. Many involve the hybridisation of a detectable probe to the amplified target, with subsequent capture washing and detection.

### SUMMARY OF THE INVENTION

The present invention provides a method for detecting nucleic acid amplification products with great facility, which does not require purification of the amplified product. Detectable product is formed without further separation steps, thus providing a homogenous assay approach.

Broadly speaking the invention is a method for detecting the product of a target- dependent nucleic acid amplification process, wherein said process uses one or more primers or probes, wherein said process produces a polydeoxyribonucleotide product when said target is RNA or produces a polyribonucleotide product when said target is DNA, comprising the steps of:

a) treating said product with a nuclease reagent whereby said product is substantially hydrolysed into its mononucleotide components, b) detecting said mononucleotide components.

In one preferred embodiment, the nuclease reagent is specific for polyribonucleotide when said product is a polyribonucleotide, and is specific for polydeoxyribonucleotide when said product is a polydeoxyribonucleotide. Most preferably, the nuclease reagent produces 5′mononucleotides.

In another preferred embodiment, the primers or probes used in the process are nuclease-resistant, or comprise a nucleic acid analogue, such as PNA.

In a further preferred embodiment, the method for detecting the mononucieotide components is specific for one or more monodeoxyribonucleotides when the product is a polydeoxyribonucleotide, and is specific for one or more monoribonucleotides when the product is a polyribonucleotide.

In a yet further preferred embodiment, the nuclease enzyme is non-specific, and hydrolyses all target, product and primer or probe polynucleotides to their component mononucieotides. In this embodiment the detection method is specific for hydrolysed product.

In further aspects the invention provides a kit for carrying out the method. Preferred embodiments of the invention may enable one to achieve one or more of the following objects and advantages: (a) To provide a universal method for detecting nucleic acid amplification products. Advantages of the present invention are that the same reagent solution may be utilised for detecting any polydeoxyribonucleotide amplification product. Similarly the same reagent solution may be utilised for detecting any polyribonucieotide amplification product.

(b) To provide a method for detecting nucleic acid amplification products that can be performed without capturing or purifying the amplification products. Advantages of the present invention are that the detection step may be performed in the same reaction vessel as was used for the amplification reaction; and the detection reaction is accomplished in a homogenous format.

(c) To provide an easy to use method for detecting nucleic acid amplification products. Advantages of the present invention is that only a moderate degree of technical skill is required bit the user.

(d) To provide an economical method for detecting the product of nucleic acid amplification reactions. An advantages of the present invention is that the components used are readily available from commercial suppliers and are relatively inexpensive.

(e) To provide a sensitive method for detecting the product of nucleic acid amplification reactions. Advantages of the present invention are that hydrolysis of the amplification product increases the number of molecules to be detected. Thus, if the product is 400 bases long, then roughly 400- fold amplification is achieved by this invention.

### BEST MODES FOR CARRYING OUT THE INVENTION

The present invention provides a method for detecting the product of a target-dependent nucleic acid amplification process, by treating said product with a nuclease reagent whereby said product is substantially hydrolysed into its mononucieotide components, and detecting said mononucleotide components. For the avoidance of doubt the meaning of the term “target-dependent nucleic acid amplification” will now be defined in general terms. Processes for the target-dependent amplification of nucleic acids involve the hybridisation of a nucleic acid probe or primer to a specific sequence in the target nucleic acid. The probe or pnmer is extended by the action of one or more enzymes to produce a complementary copy of the target sequence. This process is repeated, usually in the presence of additional enzymes and probes or pnmers, and leads to the production of many copies of nucleic to acid amplification product. The process is target-dependent because in the absence of the specific sequence in the target nucleic acid, nucleic acid amplification product is not formed. The nucleic acid amplification product comprises oligo-or poly-nucleotides, and these may comprise DNA (deoxy-ribonucleotide acid) or RNA (ribonucieotide acid). These may vary in length between 15 and 500 bases, are preferably 15-100 bases long, and are most preferably 20-30 bases in length.

The amplification process may be any process in which polynucleotides are produced in a target-specific manner from a nucleic acid target. The particular process is chosen, or modified, so that polydeoxynucieotides are produced when the target is RNA, or so that poiyribonucleotides are produced when the target is DNA.

For example, reverse transcriptase may be used in a process analogous to RT-PCR when the target is RNA. In the first round, cDNA is produced as normal by an RNA-dependent DNA synthesis using a primer. In subsequent rounds, a DNA-dependent DNA polymerase able to use either a polyribonucleotide primer, or a nuclease-resistant primer, or a nucleotide analogue primer, is used to cause DNA-directed directed synthesis of DNA, leading to a DNA product. Polyribonucleotide or nuclease-resistant primers are used, so that when the amplification product is hydrolysed using a nuclease specific for polydeoxyribonucleotides, only deoxymononucleobdes are produced.

When the target is RNA, TMA or NASBA may be utilised. These processes, which are essentially similar, use two enzymes and two primers: RNA polymerase and reverse transcriptase. One of the primers contains a promoter sequence for RNA polymerase. In the first step of amplification, the promoter-primer hybridises to the target RNA if the sequence of interest is present. Reverse transcriptase creates a DNA copy of the target RNA by extension from the 3′-end of the promoter-pnmer. The RNA in the resulting RNA:DNA duplex is degraded by the RNase H activity of the reverse transcriptase. A second primer then binds to the DNA copy. A new strand of DNA is synthesised from the end of the primer by reverse transcriptase creating a double-stranded DNA molecule. RNA polymerase recognises the promoter sequence in the DNA template and initiates transcription. Each of the newly synthesised amplicons re-enters the amplification process and serves as a template for a new round of replication leading to an exponential expansion of the RNA amplicon. Since each of the DNA templates can make 100-1000 copies of RNA amplicon, this expansion can result in the production of 10 billion amplicons in less than 1 h. In addition to the RNA amplicons, the amplification mixture will contain 5′NMP's produced through the action of RNase H on the RNA:DNA hybrids. Thus this approach does not require the addition of any additional nuclease.

Alternatively, when the target is DNA, this process may be adapted. Subsequent to the denaturation of the target DNA, a 3′-primer-promoter is hybridised to the single-stranded DNA target and is extended by reverse transcriptase in the presence of dNTP's to give a double-stranded product. This is denatured, and in the presence of a 5′ primer and dNTP's reverse transcriptase produces a further double-stranded product, which now has a promoter site for RNA polymerase. This is now cycled in the NASBA or TMA reaction: RNA polymerase recognises the promoter sequence in the DNA template and initiates transcription, producing RNA amplicons. The 5′ primer will bind to these and be extended by the reverse transcriptase to yield a DNA:RNA hybrid. Added RNase H digests the RNA strand to yield a single strand of DNA, to which the promoter primer hybridises and is extended by reverse transcriptase, to yield further copies of the double-stranded DNA having the RNA polymerase promoter site. This cycle leads to the production of up to 10 billion amplicons in less than 1 h. At the end of the amplification step, the mixture contains 5′NMP's produced by the action of Rnase H. Addition of a further nuclease specific for RNA that produces 5′NMP's may be added to increase the yield of 5′NMP's for detection.

Primers used should be of the same type as the nucleic acid target, ie when the target is DNA, the primer should be a polydeoxynucleotide, and when the target is RNA, the primer should be polyribonucleotide. However, enzymes used in the amplification process may require that the first nucleotide of the primer (the one to be extended) be of the same type as the amplification product. The invention therefore encompasses primers in which the primers are substantially comprised of the same type of nucleic acid as the target.

The nuclease reagent used may be any enzyme, which hydrolyses nucleic acids. This includes endonucleases, which are able to cleave a phosphodiester bond at any point along the polynucleotide chain; exonucleases, which are able to cleave a phosphodiester bond at the terminal ends of the polynucleotide chain; and phosphodiesterases having endo- or exo-nuclease activity. Enzymes suitable for use in the present invention include those listed in Table 1. Of these, preferred enzymes are ones that yield 5′mononucleotide hydrolysis products, and these are also indicated in Table 1.

When the target material is DNA, the nuclease reagent is chosen to be one that is specific for polyribonucteotides; when the target material is RNA, the nuclease reagent is chosen to be specific for polydeoxyribonucleotides.

Alternatively, a non-specific nuclease may be used if a method for specifically detecting the mononucleotide component resulting from the hydrolysis of the amplification product is used.

Suitably in the method, the nuclease reagent is non-specific, whereby said target and said product are substantially hydrolysed, whereby said mononucleotide components comprise deoxyrbonucleotides and ribonucleotides and wherein the step of detection is specific for the deoxyribonucleotides if the product components comprise deoxyribonucleotides or ribonucleotides if the product components comprise ribonucleotides.

TABLE 1

| EC Class | Characteristics | |
|---|---|---|
| 3. 1 4 | Phosphoric diester hydrolases | |
| | Preferred enzymes: | |
| | 3.1.4.1 Phosphodiesterase I | |
| 3 1.11 | Exodeoxynbonucleases producing 5′-phosphomonoesters | |
| | Preferred enzymes. | |
| | 3.1.11.1 Exodeoxynbonuclease I | |
| | 3.1.11.2 Exodeoxynbonuclease III | |
| | 3.1.11.3 Exodeoxynbonuclease (Lambda-induced) | |
| | 3.1.11 4 Exodeoxynbonuclease (phage Sp3-induced) | |
| | 3.1.11.5 Exodeoxynbonuclease V | |
| | 3.1.11.6 Exodeoxynbonuclease VII | |
| 3 1 13 | Exonbonucleases producing 5′-phosphomonoesters | |
| | Preferred enzymes: | |
| | 3.1.13.1 Exonbonuclease II. | |
| | 3.1.13.2 Exonbonuclease H. | |
| | 3.1.13.3 Oligonucleotidase. | |
| | 3.1.13.4 Poly(A)-specfic ribonuclease | |
| 3. 1.14 | Exonbonucleases procucing other than 5′-phospnomonoesters | |
| 3. 1.15 | Exonucleases active with either nbo- or deoxynbonucleic acid | |
| | Preferred enzymes: | |
| | 3.1.15.1 Venom exonuclease | |
| 3 1 16 | Exonucleases active witn either nbo- or deoxynbonucleic acid | |
| 3. 1.21 | Endodeoxynbonucleases producing 5-phosphomonoesters | |
| | Preferred enzymes: | |
| | 3.1.21 1 | Deoxynbonuclease |
| | 31.21.2 | Deoxynbonuclease IV (phage T4-induced) |
| 3. 1.22 | Endodeoxynbonucleases producing other than | |
| | 5′-phosphomonoesters | |
| 3. 1.25 | Site-specific endodeoxynbonucleases specific for altered bases | |
| 3. 1.26 | Endonbonucleases producing 5′-phosphomonoesters | |
| | Preferred enzymes: | |
| | 3.1.26.1 | Physarum polycephalum nbonuclease |
| 3. 1.27 | Endonbonucleases producing other than 5-phosphomonoesters | |
| 3. 1.30 | Endonucleases active with either nbo- or deoxynbonucleic | |
| | acid Preferred enzymes. | |
| | 3.1.30 1 | Aspergillus nuclease S₁ (induces mung bean |
| | | nuclease and nuclease P₁) |
| | 3.1.30.2 | Serratia marcescens nuclease |
| 3. 1.31 | Endonucleases active with either nbo- or deoxynbonucleic | |
| | acid Preferred enzymes: | |
| | 3.1.31.1 | Microccccal nuclease |

The probe or pnmer used in the process is polynbonucleotide when the target is RNA or is polydeoxyribonucieotide when the target is DNA. Alternatively the probe or pnmer may comprise PNA or other nuclease-resistant polynucteotide analogue. In further embodiments, the primer used may be resistant to the nuclease reagent used. A number of approaches for rendering oligonucteotides resistant to nuclease attack have been described, particularly in relation to the design and synthesis for resistant anti-sense oligonucleotides for use in gene therapy. To be useful in the present invention, the primers are suitably resistant to nuclease hydrolysis, yet at the same time extendable by the enzymes of the amplification process. Different cnteria for resistance apply depending on have been described, particularly in relation to the design and synthesis for resistant anti-sense oligonucleotides for use in gene therapy. To be useful in the present invention, the primers are suitably resistant to nuclease hydrolysis, yet at the same time extendable by the enzymes of the amplification process. Different criteria for resistance apply depending on whether an exonuclease (which hydrolyses the oligonucleotide in a linear fashion from one or both ends) or endonuclease (which can hydrolyse the oligonucleotide at any point along its length) are used. Where an exonuclease is used, then the presence of a single modified base in the primer will be sufficient to render the primer resitant. Examples of nuclease resistant linkages are phosphothioate and methylphosphonate linkages. These types of linkages are easily incorporated into primers.

Oligonucleotides modified so as to exhibit resistance to nucleases are known to the art. For example, Ikehara et al. (1984) Eur. J. Biochem. 139:447 reported the synthesis of a mixed octamer containing a 2′deoxy-2′-fluoroguanosine residue or a 2′-deoxy-2′-fluoroadenine residue. Ikehara et al. (1978) Nucleic Acids Res. 5:3315, showed that a 2′-chloro or bromo substituent in poly(2′deoxyadenylic acid) provided nuclease resistance. Eckstein et al. (1972) Biochemistry 11:4336, showed that poly(2′-chloro-2′-deoxyuridylic acid) and poly(2′-chloro-2′-deoxycytidylic acid) are resistant to various nucleases. Inoue et al. (1987) Nucleic Acids Res. 15:6131, described the synthesis of mixed oligonucleotide sequences containing 2′-OCH.sub.3 at every nucleotide unit. The mixed 2′OCH3substituted sequences hybridized to their RNAs as strongly at the non-substituted RNAs. Shibahara et al. (1987) Nucleic Acids Res. 17:239, also described the synthesis of mixed oligonucleotide sequences containing 2′-OCH₃ at every nucieotide unit. The stability of oligoribonucleotides against endonuclease degradation may be achieved by replacement of the 2′-OH group of the ribose moiety with an alternate substituent such as an amino group or a fluoro group. Both 2′-amino and 2′-fluoro nucleoside 5-triphosphates are substrates for T7 RNA polymerase, albeit with somewhat decreased incorporation efficiency (Aurup et al. (1992) Biochemistry 31:9636-9641). Other 2′-substituted nucleotides such as 2′-O-methyl, 2′-O-alkyl, or 2′-deoxy nucleoside 5-triphosphates are not recognized as substrates by T7 RNA polymerase.

However, modifications at the phosphorous atom of the oligonucleotide, while exhibiting various degrees of nuclease resistance, have generally suffered from inferior hybridisation properties [Cohen, J. S., Ed., Oligonucleotides. Antisense Inhibitors of Gene Expression (CRC Press, Inc., Boca Raton, Fla., 1989)].

To enhance hybridisation fidelty, phosphorothioate oligonucleotides having substantially chirally pure intersugar linkages are greatly desired. Further, such phosphorothioate oligonucleotides having substantially chirally pure intersugar linkages would lead to more efficacious therapeutic compounds. In U.S. Pat. No. 5599797, phosphorothioate oligonucleotides having all nucleoside units joined together by either substantially all Sp phosphorothioate intersugar linkages or substantially all Rp phosphorothioate intersugar linkages are provided.

Replacement of the phosphorus atom has been an alternative approach in attempting to avoid the problems associated with modification on the pro-chiral phosphate moiety, and methods for preparing such analogues are disclosed in U.S. Pat. No. 5,618,704.

U.S. Pat. No. 5,672,697 describes novel methylene phosphonate nucieosides and novel oligonucleotides derived from them that have enhanced nuclease stability.

U.S. Pat. No. 5,705,333 describes chimeric PENAMs (peptide-based nucleic acid mimic), which have an unusual stereochemical composition that facilitates binding to the target nucleic acid. In particular, they have a peptidic backbone that incorporates unusual chiral centres (including D-chiral centres and quasi-chiral centres) that can be used to orient the nucleic side chains in such a way that the nucleotidic bases are spatially homomorphic to bases in targeted nucleic acids. The ability to enhance binding by spatial homomorphism is especially significant given that hydrogen-bonding interactions between biomolecules typically depend on an aggregation of many relatively weak bonds. The PENAMs are also much less susceptible to electrostatic charge repulsion (because of the replacement of the normally charged backbone). Also, by virtue of their unusual structural and stereochemical features, the PENAMs of the present invention are resistant to degradative enzymes that are expected to be present in most biological systems. In particular, the PENAMs do not possess the phosphodiester backbone that is the standard target of the nucleases. Moreover, the peptidic backbone is unlike that of naturally occurring peptides because of the presence of unusual chiral centres including D-chiral centers and/or quasi-chiral centers.

U.S. Pat. No. 5,612,458 to Hyidig-Nielson and Pluzek, uses peptide nucleic acid (PNA) resistant to nuclease.

The mononucleotide hydrolysis products may be detected by a number of means. Where the mononucleotide hydrolysis products are 5′mononucieotides, a preferred method of detection involves converting the 5′mononucieotides to 5′ADP using nucleoside monophosphate kinase (E.C.2.7.4.4). Alternatively, adenylate kinase (E.C. 2.7.4.3) or guanylate kinase (E.C. 2.7.4.8) may be used to convert 5′AMP or 5′GMP to 5′ADP.

Table 2 summarises some of the enzymes able to transfer a phosphate group from ATP to a 5′NMP to give ADP.

TABLE 2

| EC | | |
|---|---|---|
| number | Enzyme | NMP |
| 2.7.4.3 | adenylate kinase | AMP |
| 2.7.4.4 | nuclecside-phosphate kinase | NMP |
| 2.7.4.8 | guanylate kinase | (d)GMP |
| 2.7.4.9 | thymidylate kinase | TMP |
| 2.7.4.10 | nucleoside-triphosphate adenylate kinase | AMP |
| 2.7.4.11 | deoxyadenylate kinase | (d)AMP |
| 2.7.4.13 | deoxynucleoside-phospnate kinase | (d)NMP |
| 2.7.4.14 | cytidylate kinase | (d)CMP |

A number of methods for measuring 5′ADP are known in the art. For example, pyruvate kinase (E.C. 2.7.1.40) will catalyse the transfer of a phosphate group from to phosphoenol pyruvate to ADP to yield pyruvate and ATP. Pyruvate is a substrate for pyruvate oxidase (E.C. 1.2.3.3), which catalyses its hydrolysis, yielding hydrogen peroxide, which is detected using, for example, horseradish peroxidase in a colorimetric, fluorimetric or luminometric manner.

Alternatively, pyruvate may be reduced to lactate in the presence of NADH and the enzyme lactate dehydrogenase. Lactate produced is a substrate for lactate oxidase (E.C. 1.13.12.4), which catalyses its hydrolysis, yielding hydrogen peroxide, which is detected using, for example, horseradish peroxidase in a colorimetric, fluorimetric or luminometric manner.

Particularly attractive applications, which illustrate the operation of the present invention, are described below.

Referring now to Schematic 1, at the end of the description, which shows particularly preferred embodiments of the present invention, DNA targets are amplified to polyribonucleotide products and RNA targets are amplified to deoxyribonucleotide products.

The left hand side of Schematic I shows treatment of the product with a specific nuclease that preferably produces 5′NMN's from polyribonucleotide products, and 5′dNMN's from polydeoxyribonucleotide products. Where the product is polydeoxyrbonucleotide, DNase I (EC 3.1.21.1) may be used as the specific enzyme, at a pH of 6.5 to 7.0. Where the product is polyribonucieotide, exoribonuclease 11 (EC3.1.13.1) may be utiiised.

These are converted to 5′ADP using one or more of the enzymes listed in Table 2, typically at a pH of 6.7 to 7.0.

The right hand side of Schematic 1 shows treatment of the reaction mixture with a non-specific nuclease, which yields a mixture of 5′dNMP's and 5′NMP's. The non-specific nuclease may be Nuclease P, at about pH 6.0. An enzyme specific either for 5′dNMP's (when the product is a polydeoxyribonucleotide) or 5′NMP's (when the product is a polyribonucleotide) are used to yield ADP.

ADP is treated with pyruvate kinase and phosphoenol pyruvate at about pH 6-7, to yield pyruvate. Pyruvate oxidase is used to convert pyruvate to acetyl phosphate and hydrogen peroxide at about pH 6-7. Hydrogen peroxide can be detected colorimetrically, iuminometrically or fluorimetrically using horseradish peroxidase. Altematively, an acceptor such as dichlorophenol indolphenol may be used in the lactate oxidase reaction instead of oxygen, leading to the formation of a coloured material directly.

### EXAMPLES

1. RT-PCR amplification converts RNA target to DNA product

The following mixture is prepared: 4 μl 25 mM MgCl₂, 2 μl 0.1 M Tris-HCI pH 9.0 containing 0.5 M KCI and 1% Triton X-100, 2 μl 10 mM dNTP mixture, 15 U AMV reverse transcriptase, 50 pmol 3′-primer and DNA-free RNA target in a total volume of 20 μl. After incubation at 42° C. for 15 minutes, AMV reverse transcnptase is inactivated by heating at 99° C. for 5 minutes, followed by cooling at 0-5° C. for 5 minutes. The resulting solution is mixed with 80 μl nuclease-free water, and 10 μl of this is added to the following mixture for PCR: 4 μl 25 mM MgCl₂, 8 μl 0.1 M Tris-HCI pH 9.0 containing 0.5 M KCI and 1% Triton X-100, 2 μl 10 mM dNTP′ mixture, 50 pmol 5′-primer, 50 pmol 3′ primer, and 2.5 U Taq DNA polymerase in a total volume of 100 μl. Typically 15-40 PCR cycles are conducted. The primers used are RNA primers or nuclease-resistant primers.

2. NASBA, TMA amplification converts DNA target to RNA product

Denatured RNA-free DNA (5 μl) target is mixed with 10 μl of a mixture comprising 40 mM Tris-HCI pH 8.5,12 mM MgCl₂, 70 mM KCI, 5 mM DOTT, 1 mM dNTP mixture, 2 mM each NTP mixture, 15% DMSO, 0.2 μM of the promoter-primer and 6.4 U AMV reverse transcriptase. After incubation at 42° C. for 15 minutes, AMV reverse transcriptase is inactivated by heating at 99° C. for 5 minutes, followed by cooling at 0-5° C. for 5 minutes. To this is added 5 ha of an mixture comprising 1.5 M sorbitol, 2.1 μg BSA, 0.6 4M of the second primer, 32 T7 RNA polymerase, 6.4 U AMV reverse transcriptase and 0.08 U RNase H to give a total volume of 20 μl. Isothermal amplification is performed at 41° C. for 1.5 h. The pnmers used are DNA or are nuclease-resistant.

3. DNA product is converted to 5′dNMP's To 10 μg) of amplification mixture is added 10 μl of a solution compnising 50 mm sodium acetate buffer, 20 mM MgCl₂, 2 mM OTT, 0.5 mg RNase-free DNAse I, and the mixture incubated for 15 minutes at 37° C.

4. RNA product is converted to SINMP's

To 10 μl of amplification mixture is added 10 μlof a solution comprising 50 mM sodium acetate buffer, 20 mM MgCl₂, 0.5 mg DNas0m free physarum polycephanum ribonuctease, and the mixture incubated for 15 minutes at 37° C.

5. Amplification product is converted to 5′NMP's and 5′dNMP's

To 10 μl of amplification mixture is added 10 μl of a solution comprising 50 mM sodium acetate buffer, 20 mM MgCl2, 2 mM DTT, 0.5 mg RNasefree DNAse I, and incubate the mixture for 15 minutes at 37° C.

6. 5′NMP's are converted to pyruvate and detected using lactate dehydrogenase

10 μl of 5′NMP's from 3 above added to 90 μl of a solution containing: 8.5 mM ATP, 1.22 mM NADH, 2.0 mM PEP, 7.0 U/ml nucleoside monophosphate kinase, 15.3 u/ml Lactate Dehydrogenase, Pyruvate kinase 7.0 u/ml, 28.0 mM MgSO₄,7H₂O, 26.0 mM Reduced Glutathione, 50 mM HEPES buffer 7.4. The concentration of 5′NMP's is determined from the change in absorbance at 340 nm.

7. 5′NMP's are converted to pyruvate and detected using pyruvate oxidase

10 μl of 5′NMP's from 3 above added to 90 μl of a solution containing: 8.5 mM ATP, 2.0 mM PEP, 7.0 U/ml nucleoside monophosphate kinase, 7.0 u/mi pyruvate kinase, 1.0 U/ml pyruvate oxidase, 60 μg horseradish peroxidase, 0.2 mM 4-amino- antipyrine, 2 mM 3,5-dichloro-2-hydroxy-benzene sulphonic acid, 28.0 mM MgSO,.7H₂O, 50 mM Mes buffer 6.0. The concentration of 5′NMP's is determined from the change in absorbance at 520 nm.

8. 5′dNMP's are converted to pyruvate and detected using lactate dehydrogenase

10 μl of 5′NMP's from 3 above added to 90 μl of a solution containing: 8.5 mM ATP, 1.22 mM NADH, 2.0 mM PEP, 7.0 U/ml each of adenylate kinase, guanylate kinase, and cytidyiate kinase, 15.3 U/ml Lactate Dehydrogenase, Pynivate kinase 7.0 u/ml, 28.0 mM MgSO₄.7H₂O, 26.0 mM Reduced Glutathione, 50 mM HEPES buffer 7.4. The concentration of 5′NMP's is determined from the change in absorbance at 340 nm.

9. 5′NMP's are converted to pyruvate and detected using pyruvate oxidase

10 μl of 5′NMP's from 3 above added to 90 μl of a solution containing: 8.5 mM ATP, 2.0 mM PEP, 7.0 U/ml each of adenylate kinase, guanylate kinase, and cytidyiate kinase, 7.0 U/ml pyruvate kinase, 1.0 U/ml pyruvate oxidase, 60 μg horseradish peroxidase, 0.2 mM 4-amino-antipyrine, 2 mM 3,5-ichioro-2-hydroxy-benzene sulphonic acid, 28.0 mM MgSO₄.7H₂O, 50 mM Mes buffer 6.0. The concentration of 5′NMP's is determined from the change in absorbance at 520 nm.

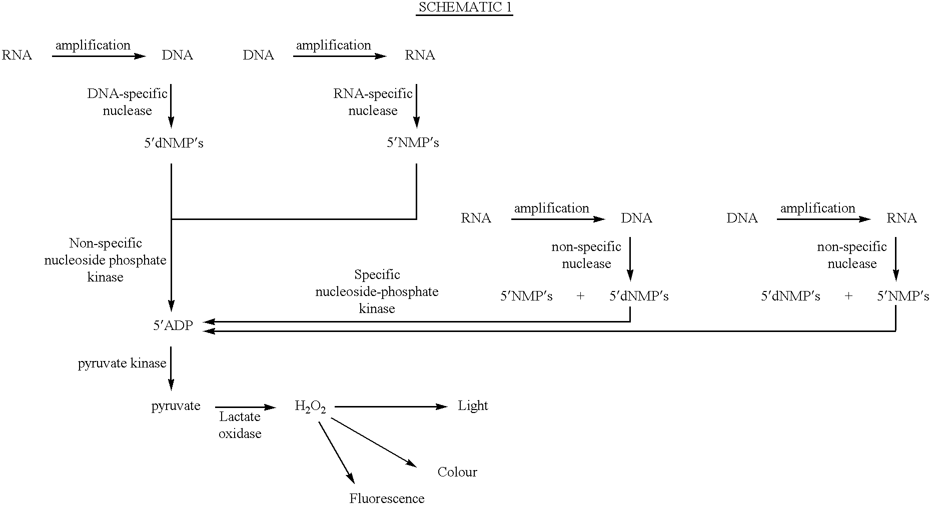

1. A method for detecting nucleic acid amplification product of a target-dependent nucleic acid amplification process involving one or more probes or primers, comprising the steps of:
a) treating said product with a nuclease reagent whereby said product is substantially hydrolysed into its mononucleotide components,
b) detecting said mononucieotide components.

2. The method of claim 1 wherein said product is a polydeoxyribonucleotide product when said target is RNA or is a polyribonucleotide product when said target is DNA.

3. The method of claim 2 wherein said nuclease reagent is specific for polyribonucieotide when said product is a polyribonuctectide, and is specific for polydeoxyribonucleotide when said product is a polydeoxyribonucleotide.

4. The method of claim 1, 2 or 3 wherein the or each said probe or primer is nuclease-resistant.

5. The method of claim 1, 2, 3 or 4 wherein said probe or primer comprises a nucleic acid analogue.

6. The method of claim 5 wherein said nucleic acid analogue comprises PNA or PENAM.

7. The method of any preceding claim wherein said target-dependent nucletc acid amplification process is selected from the group consisting of: RT-PCR, PCR, SDA, TMA, and NASBA.

8. The method of any preceding claim wherein said mononucleotide components are converted to 5′ADP and said 5′ADP is detected.

9. The method of claim 8 wherein said mononucleotide components are converted to 5′ADP in a reaction catalysed by a kinase from Enzyme Commission class 2.7.4, and said 5′ADP is detected.

10. The method of claims 8 or 9 wherein said 5′ADP is detected by additional steps comprising:
b. converting said 5′ADP to pyruvate by means of pyruvate kinase in the presence of phosphoenol pyruvate,
c. converting said pyruvate to hydrogen peroxide by means of pyruvate oxidase in the presence of oxygen and phosphate,
e. detecting said hydrogen peroxide.

11. The method of claim 9 wherein said 5′ADP is detected by additional steps comprising:
b. converting said 5′ADP to pyruvate by means of pyruvate kinase in the presence of phosphoenol pyruvate,
c. converting said pyruvate to lactate by means of lactate dehydrogenase in the presence of NADH,
e. detecting a change in the absorbance of said NADH.

12. The method of claim 1 wherein said target-dependent nucleic acid amplification process comprises a step where the RNA portion of a DNA:RNA hybrid is hydrolysed to mononucleotide components, and wherein the step where said DNA:RNA hybrid is hydrolysed is catalysed by said nuclease reagent.

13. The method of claim 12 wherein said target-dependent nucleic acid amplification process is TMA or NASBA.

14. The method of claim 12 wherein said nuclease reagent is RNase H.

15. The method of claim 12 wherein said nuclease reagent is an RNA polymerase enzyme that also has RNase H activity.

16. The method of claim 2 wherein said nuclease reagent is non-specific, whereby said target and said product are substantially hydrolysed, whereby said mononucleotide components comprise deoxyribonucieotides and ribonucleotides and wherein the step of detection is specific for the deoxyribonucleotides if the product components comprise deoxyribonucleotides or ribonucieotides if the product components comprise ribonucleotides.

17. The method of claim 16 wherein said target is RNA and said product is polydeoxyribonucleotide, and wherein for detection said deoxyribonucleotides are converted to 5′ ADP in a reaction by a Kinase that is specific for deoxyribonucieotides.

18. The method of claim 16 wherein said target is DNA and said product is a polyribonucleotide, and wherein for detection said ribonucleotides are converted to 5′ ADP in a reaction catalysed by a kinase that is specific for ribonucleotides.
